(19) United States
(12) Patent Application Publication
(10) Pub. No.: US 2025/0333373 A1
(43) Pub. Date: Oct. 30, 2025

(54) L-2-Amino-4-Halobutyric Acid Derivative-L-Tartrate and Preparation Method Thereof

(71) Applicant: Vulpes Agricultural Corp., St. Louis, MO (US)

(72) Inventors: Dihu Yu, St. Louis, MO (US); Rick Shang, St. Louis, MO (US)

(73) Assignee: Vulpes Agricultural Corp., St. Louis, MO (US)

(21) Appl. No.: 18/866,255

(22) PCT Filed: May 23, 2023
(86) PCT No.: PCT/US2023/023205

§ 371 Date: Nov. 18, 2024

Related U.S. Application Data
(60) Provisional application No. 63/345,296, filed on May 24, 2022.

(51) Int. Cl. C07C 229/20; C07C 59/255

(52) U.S. Cl. CPC C07C 229/20; C07C 59/255

(57) ABSTRACT

The present disclosure describes an L-S2-amino-4-halobutyric acid derivative-L-tartrate salt. The present disclosure also describes a method of preparing L-S-amino-4-halobutyric acid derivative-L-tartrate salt, comprising the following steps: using D, L-S2-amino-4-halobutyric acid derivative enantiomeric mixture as the raw material, using L-tartrate as the resolution agent, and obtaining L-S2-Amino-4-halobutyric acid derivative-L-tartrate by resolution reaction, crystallization and filtration in the same resolution agent system; or the enantiomeric mixture of D, L-S2-amino-4-halobutyric acid derivatives is used as raw material, L-tartrate is used as resolution agent under aromatic aldehyde catalysis or aromatic aldehyde and organic acid co-catalysis, and dynamic resolution reaction is carried out in the same resolution agent system, further comprising crystallization and filtration to obtain L-S2-amino-4-halobutyric acid derivative-L-tartrate.

### FIELD OF THE INVENTION

This disclosure relates to L-2-amino-4-halobutyric acid derivative-L-tartrate and methods of its preparation. This compound is a chiral intermediate that can be used in the production of the herbicide L-glufosinate.

### BACKGROUND OF THE INVENTION

In 1987, the German company Hoechst pioneered the development of glufosinate herbicide and successfully commercialized it under the trade name Basta.

The most used glufosinate is the racemate of L and D glufosinate, of which L-glufosinate has an herbicidal effect while D is almost inactive. Because L-glufosinate has twice the herbicidal activity of common glufosinate, the application amount is only 50% of glufosinate, and the application cost is basically the same. The development and production of L-glufosinate will greatly reduce the amount of glufosinate use, which is very important to improve the cost-effectiveness of the product, reduce the amount of pesticide use, and reduce environmental pressure.

Commercialized glufosinate is mainly D, L-glufosinate, which is mainly produced by the following methods.

U.S. Pat. No. 4,599,207A uses a thermal cracking-ACA process to prepare methylphosphine dichloride (MDP) by the gas-phase reaction of methane and phosphorus trichloride. The most important steps are the preparation of the key block “P” group methyl phosphite monobutyl ester (MPE) and the Michael radical addition of the amino butyric acid precursor 2-acetoxy-3-butenenitrile (ACA), followed by ammonification and hydrolysis to obtain glufosinate.

Patent CN102276643B uses the Grignard-Strecker process in order to bypass the step of producing MDP. Diethyl methyl phosphite is obtained as a “P” group block by means of Grignard's reagent, and then reacted with acrolein, the precursor of the aminobutyric acid block, to obtain aminonitrile by Strecker reaction, which is hydrolyzed to obtain glufosinate.

Patent application CN106046052A, based on the previous patent, uses the Al-Strecker process. The MDP is produced by complexing and uncomplexing phosphorus trichloride, chloromethane, and aluminum trichloride, and then diethyl methylphosphite is synthesized as a “P” group block. The subsequent steps are the same as in the Grignard-Strecker process, but the advantage is that the MDP can be synthesized and the latter part can be changed to the ACA process, so the choice of aminobutyric acid blocks is more flexible.

Patent application CN110003269A uses methane and phosphorus trichloride as raw materials for the preparation of MDP and subsequent “P” group blocks, without Grignard's reagent or aluminum trichloride.

In general, two key blocks are required for the preparation of glufosinate, namely the “P” block (e.g., monobutyl methyl phosphite, diethyl methyl phosphite, etc.) and the amino butyric acid block (e.g., ACA, acrolein, etc.). At present, the synthesis of D, L-glufosinate has been very mature in the industrialization of “P” group blocks.

At present, research into the production of L-glufosinate is mainly divided into chemical resolution, asymmetric chiral synthesis, biological enzyme method and chiral source method.

The chemical resolution mainly targets D, L-glufosinate.

Patent U.S. Pat. No. 5,767,309 discloses the resolution method of D, L-glufosinate, which utilizes quinine and glufosinate-ammonium to form a salt, and then utilizes 3,5-dinitrosalicylaldehyde to racemize in the presence of acetic acid to obtain L-glufosinate. However, the yield is low and quinine is expensive, so this process has not been industrialized.

Patent application WO2018108797A1 discloses a method for the preparation of L-glufosinate using ephedrine analogues to induce crystallization. The method allows the conversion of D-glufosinate to L-glufosinate. The structure of ephedrine analogues is simple, but ephedrine belongs to a category of controlled substance, so the process is unlikely to be industrialized.

Patent application CN112979701A discloses a method for the preparation of L-glufosinate. D, L-glufosinate derivatives are reacted with ligands or their hydrochloride and nickel salts in the presence of alkali to obtain metal complexes, which are then hydrolyzed to obtain L-glufosinate. The process route is complicated, with more steps, lower splitting efficiency, and higher cost of ligands involved, and is not easy to industrialize.

Asymmetric chiral synthesis starts with the synthesis of a non-chiral phosphine compound precursor followed by asymmetric synthesis.

Patent application WO2006104120 discloses a method for the asymmetric hydrogenation of dehydroamino acids using a rhodium catalyst to obtain L-glufosinate by hydrolytic conversion. The route uses acrylate as the precursor block of aminobutyric acid, reacts with the “P” group block, then hydrolyzes and ammonizes it, and then constructs the chiral center by asymmetric hydrogenation with mild reaction conditions and high yield. However, hydrogenation requires chiral phosphorus ligands for rhodium catalyst hydrogenation, which is expensive, difficult to recover and costly to produce.

Patent application WO2008035687 discloses a Jacobsen catalyst-catalyzed synthesis of L-glufosinate. The β-hypophosphoryl aldehyde obtained from the reaction of the above two blocks was reacted with aromatic amines to form imine compounds, which were catalyzed by Jacobsen catalyst to carry out asymmetric strecker reaction with trimethylsilyl cyanide to obtain L-glufosinate by hydrolytic conversion, but both the raw material trimethylsilyl cyanide and Jacobsen catalyst are costly and difficult to industrialize.

Patent application CN105131032A discloses a method for synthesizing L-glufosinate using cinchonidine chiral quaternary ammonium salt derivatives as phase transfer catalysts. The patent application uses benzylidene glycine ester compounds as aminobutyric acid blocks and methyl vinyl phosphonate compounds as “P” blocks. Under the catalysis of chiral catalysts, the chiral center of L-glufosinate is constructed by asymmetric Michael addition, which gives L-glufosinate after hydrolysis. However, the catalyst consumption of this route is large and difficult to recover; the e.e. value and yield are low.

Biological enzymatic methods are mainly divided into biological enzyme resolution and transaminase methods.

Patents and patent applications U.S. Pat. Nos. 5,618,728, 5,756,800, CN105567780, CN107502647, U.S. Pat. No. 9,834,802B2, CN109384811A all disclose methods for preparing L-glufosinate by resolution or conversion with biological enzymes using D, L-glufosinate or its derivatives as raw materials, but all have problems such as unused D-glufosinate, complex systems or difficulties in catalyst recovery.

JP2589693B2, CN110093327A, and CN106916857A all disclose the process of converting keto groups into amino groups by transaminase to obtain L-glufosinate. The patent uses a keto acid, such as 2-carbonyl-4-(hydroxymethylphosphono) butyric acid, as a substrate and L-glutamic acid as a chiral amino donor, but there is also the problem of difficulty in product purification.

Biological enzymatic production of L-glufosinate, neither of which involves the preparation and reaction associated with the two aforementioned blocks, has strict environmental requirements on microorganisms and enzymes. There is significant high phosphorus-containing wastewater, the cost is not easy to control, the system is complex and the product purification is difficult, so few industrially produced commodities are available.

The chiral source method synthesizes L-glufosinate by preparing a chiral aminobutyric acid block and reacting it with a “P” group block.

Patent U.S. Pat. No. 5,591,861A discloses the method of preparing chiral aminobutyric acid blocks using L-glutamic acid and L-aspartic acid as chiral sources.

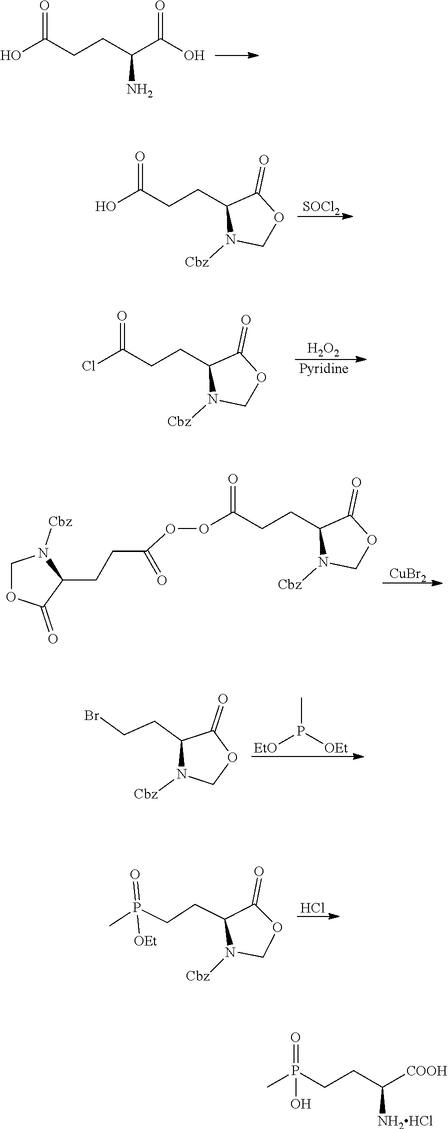

The raw materials are protected and acylated to obtain β-haloethyl-L-glycine derivatives, and then reacted with diethyl methyl phosphite by Arbuzov reaction to obtain L-glufosinate derivatives, which are further hydrolyzed to obtain L-glufosinate hydrochloride. This route has more steps and requires multi-step conversion, which is difficult to industrialize.

CN106083922A discloses a method for preparing chiral aminobutyric acid blocks of 4-halo-2-aminobutyrate from L-methionine. L-methionine is reacted with α-halogenated carboxylic acid or its derivatives under the action of phase transfer catalyst to obtain L-homoserine lactone hydrogen halide salt, and then L-glufosinate is obtained by subsequent reaction. However, sulfur pollution is serious.

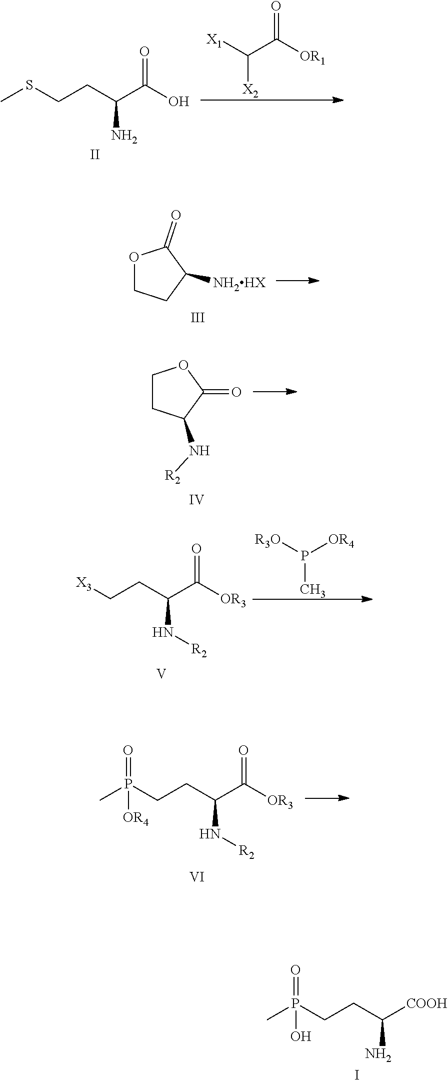

Patents and patent applications U.S. Pat. No. 5,442,088, CN111662324A, CN112574119A, CN110845347A, WO2021147894A1, WO2021143713A1, and CN109369432A disclose the preparation of chiral aminobutyric acid block 4-halogenated-2-aminobutyric acid (ester) from L-homoserine or its endolipid hydrochloride. However, the L-homoserine method has problems such as expensive raw materials, serious pollution, harsh ring-opening conditions, and the need for special reaction equipment, making it difficult to industrialize.

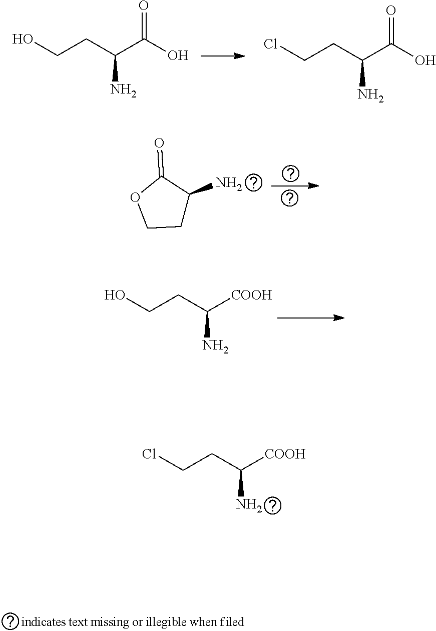 [SMILES: N[C@@H](CCO)C(=O)O ; N[C@@H](CCCl)C(=O)O ; N[C@H]1CCOC1=O ; N[C@@H](CCO)C(=O)O ; N[C@@H](CCCl)C(=O)O]

Patent applications WO2006103696A2 and CN102060721A split aminobutyramide and aminobutyric acid by tartaric acid respectively, but aminobutyramide and aminobutyric acid cannot be directly used as intermediates for the synthesis of L-glufosinate, and their purpose is not to prepare L-glufosinate.

As mentioned above, the industrial synthesis of D,L-glufosinate from “P” blocks and aminobutyric acid blocks is well established. However, the key to the preparation of L-glufosinate lies in the availability of chiral intermediates of aminobutyric acid blocks at low cost. From the current public reports, there is still no route that can be used to prepare chiral aminobutyric acid blocks for the synthesis of L-glufosinate in an efficient, green and simple way. In particular, there is no method to obtain chiral intermediates of L-glufosinate by the resolution of racemic aminobutyric acid or its derivatives. Therefore, a low-cost, simple route and industrially feasible synthesis process of chiral aminobutyric acid blocks is urgently needed.

### BRIEF SUMMARY OF THE INVENTION

One aspect of the disclosure is directed to an L-2-amino-4-halobutyric acid derivative-L-tartrate with a structure of formula I:

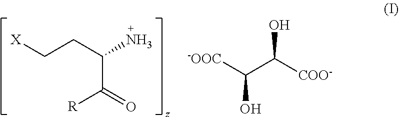

wherein
z is the molar ratio of L-2-amino-4-halobutyric acid derivative to L-tartaric acid, z is from 1 to 2;
X is Cl or Br; and
R is OR¹ or NR²R³, wherein R¹ is a C₁ to C₄ aliphatic hydrocarbon, R² is H or a C₁ to C₄ aliphatic hydrocarbon, and R³ is H or a C₁ to C₄ aliphatic hydrocarbon.

Another aspect of the disclosure is a method of preparing a L-2-amino-4-halobutyric acid derivative-L-tartaric acid salt, described as method a) elsewhere in the disclosure, the method comprising:
a) mixing a D, L-2-amino-4-halobutyric acid derivative enantiomeric mixture, L-tartaric acid, and a solvent to facilitate a resolution reaction and produce a resolution reaction mixture comprising L-2-amino-4-halobutyric acid derivative-L-tartrate;
b) precipitating the L-2-amino-4-halobutyric acid derivative-L-tartrate; and
c) separating the L-2-amino-4-halobutyric acid derivative-L-tartrate from the resolution reaction mixture.

A further aspect of the disclosure is a method of preparing a L-2-amino-4-halobutyric acid derivative-L-tartaric acid salt, described as method b) elsewhere in the disclosure, the method comprising:
a) mixing a D-2-amino-4-halobutyric acid derivative or a D, L-2-amino-4-halobutyric acid derivative enantiomeric mixture, L-tartaric acid, aromatic aldehydes or aromatic aldehydes and organic acids, and a solvent to facilitate a dynamic resolution reaction and produce a resolution reaction mixture comprising L-2-amino-4-halobutyric acid derivative-L-tartrate;
b) precipitating the L-2-amino-4-halobutyric acid derivative-L-tartrate; and
c) separating the L-2-amino-4-halobutyric acid derivative-L-tartrate from the resolution reaction mixture.

Other objects and features will be in part apparent and in part pointed out hereinafter.

### BRIEF DESCRIPTION OF THE SEVERAL VIEWS OF THE DRAWING

FIG. 1 shows the infrared spectrum of L-2-amino-4-chlorobutyric acid ethyl ester-L-tartrate prepared in Example 1 of the present invention.

FIG. 2 shows the NMR hydrogen spectrum of L-2-amino-4-chlorobutyric acid ethyl ester-L-tartrate prepared in Example 1 of the present invention.

FIG. 3 shows the NMR carbon spectrum of L-2-amino-4-chlorobutyric acid ethyl ester-L-tartrate salt prepared in Example 1 of the present invention.

FIG. 4 shows the infrared spectrum of ethyl L-2-amino-4-bromobutyrate-L-tartrate prepared in Example 6 of the present invention.

FIG. 5 shows the NMR hydrogen spectrum of L-2-amino-4-bromobutyric acid ethyl ester-L-tartrate prepared in Example 6 of the present invention.

FIG. 6 shows the NMR carbon spectrum of L-2-amino-4-bromobutyric acid ethyl ester-L-tartrate salt prepared in Example 6 of the present invention.

FIG. 7 shows the infrared spectrum of L-2-amino-4-chlorobutyric acid methyl ester-L-tartrate prepared in Example 8 of the present invention.

FIG. 8 shows the NMR hydrogen spectrum of L-2-amino-4-chlorobutyric acid methyl ester-L-tartrate prepared in Example 8 of the present invention.

FIG. 9 shows the NMR carbon spectrum of L-2-amino-4-chlorobutyric acid methyl ester-L-tartrate salt prepared in Example 8 of the present invention.

FIG. 10 shows the infrared spectrum of isopropyl L-2-amino-4-chlorobutyrate-L-tartrate prepared in Example 10 of the present invention.

FIG. 11 shows the NMR hydrogen spectrum of isopropyl L-2-amino-4-chlorobutyrate-L-tartrate prepared in Example 10 of the present invention.

FIG. 12 shows the NMR carbon spectrum of isopropyl L-2-amino-4-chlorobutyrate-L-tartrate prepared in Example 10 of the present invention.

FIG. 13 shows the infrared spectrum of L-2-amino-4-chlorobutyramide-L-tartrate prepared in Example 12 of the present invention.

FIG. 14 shows the NMR hydrogen spectrum of L-2-amino-4-chlorobutyramide-L-tartrate prepared in Example 12 of the present invention.

FIG. 15 shows the NMR carbon spectrum of L-2-amino-4-chlorobutyramide-L-tartrate prepared in Example 12 of the present invention.

Corresponding reference characters indicate corresponding parts throughout the drawings.

### DETAILED DESCRIPTION OF THE INVENTION

One aspect of the disclosure is a L-2-amino-4-halobutyric acid derivative-L-tartrate with a structure of formula I.

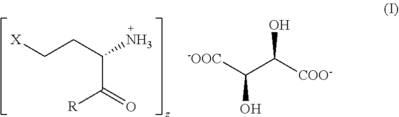

wherein
z is the molar ratio of L-2-amino-4-halobutyric acid derivative to L-tartaric acid, z is from 1 to 2;
X is Cl or Br; and
R is OR¹ or NR²R³, wherein R¹ is a C₁ to C₄ aliphatic hydrocarbon, R² is H or a C₁ to C₄ aliphatic hydrocarbon, and R³ is H or a C₁ to C₄ aliphatic hydrocarbon.

Another aspect of the disclosure is a method of preparing a L-2-amino-4-halobutyric acid derivative-L-tartaric acid salt, described as method a) elsewhere in the disclosure, the method comprising:
a) mixing a D, L-2-amino-4-halobutyric acid derivative enantiomeric mixture, L-tartaric acid, and a solvent to facilitate a resolution reaction and produce a resolution reaction mixture comprising L-2-amino-4-halobutyric acid derivative-L-tartrate;
b) precipitating the L-2-amino-4-halobutyric acid derivative-L-tartrate; and
c) separating the L-2-amino-4-halobutyric acid derivative-L-tartrate from the resolution reaction mixture.

The molar ratio of L-2-amino-4-halobutyric acid derivatives to D-2-amino-4-halobutyric acid derivatives in the D, L-2-amino-4-halobutyric acid derivative enantiomeric mixture can be from 0.5 to 1.5:1. The molar ratio of the enantiomeric mixture of D, L-2-amino-4-halobutyric acid derivatives to L-tartaric acid can be from 1:0.2 to 1.5.

The solvent can be selected from one or more C₁ to C₄ lower alcohols or a mixture of one or more C₁ to C₄ lower alcohols with water; the C₁ to C₄ lower alcohol being one or more of methanol, ethanol, n-propanol, isopropanol, n-butanol, isobutanol, and tert-butanol; and the mixture of one or more C₁ to C₄ lower alcohols with water having a volume percentage of C₁ to C₄ lower alcohols from 10% to 99.99%. The volume of the solvent can be from 1 mL/g D, L-2-amino-4-halobutyric acid derivative enantiomeric mixture to 30 mL/g D, L-2-amino-4-halobutyric acid derivative enantiomeric mixture. The temperature of step a) of the method can be from 0° C. to the reflux temperature of the system.

A further aspect of the disclosure is a method of preparing a L-2-amino-4-halobutyric acid derivative-L-tartaric acid salt, described as method b) elsewhere in the disclosure, the method comprising:
a) mixing a D-2-amino-4-halobutyric acid derivative or a D, L-2-amino-4-halobutyric acid derivative enantiomeric mixture, L-tartaric acid, aromatic aldehydes or aromatic aldehydes and organic acids, and a solvent to facilitate a dynamic resolution reaction and produce a resolution reaction mixture comprising L-2-amino-4-halobutyric acid derivative-L-tartrate;
b) precipitating the L-2-amino-4-halobutyric acid derivative-L-tartrate; and
c) separating the L-2-amino-4-halobutyric acid derivative-L-tartrate from the resolution reaction mixture.

The molar ratio of L-2-amino-4-halobutyric acid derivatives to D-2-amino-4-halobutyric acid derivatives in the D, L-2-amino-4-halobutyric acid derivative enantiomeric mixture can be from 0 to 1.5:1. The molar ratio of the D-2-amino-4-halobutyric acid derivative or the enantiomeric mixture of D, L-2-amino-4-halobutyric acid derivatives to L-tartaric acid can be from 1:0.3 to 1.5.

The aromatic aldehydes can be one or more of benzaldehyde, salicylaldehyde, 5-nitrosalicylaldehyde, and 3,5-dinitrosalicylaldehyde. The molar ratio of the D-2-amino-4-halobutyric acid derivative or the enantiomeric mixture of D, L-2-amino-4-halobutyric acid derivatives to the aromatic aldehyde can be from 1:0.01 to 0.5.

The organic acids can be one or more of formic acid, acetic acid, and propionic acid. The molar ratio of the D-2-amino-4-halobutyric acid derivative or the enantiomeric mixture of D, L-2-amino-4-halobutyric acid derivatives to the organic acid can be from 1:0.1 to 5.

The solvent can be selected from one or more C₁ to C₄ lower alcohols or a mixture of one or more C₁ to C₄ lower alcohols and water; the C₁ to C₄ lower alcohol being one or more of methanol, ethanol, n-propanol, isopropanol, n-butanol, isobutanol, and tert-butanol; and the mixture of one or more C₁ to C₄ lower alcohols and water having a volume percentage of C₁ to C₄ lower alcohols from 80% to 99.99%. The volume of the solvent can be from 1 mL/g D-2-amino-4-halobutyric acid derivative or D, L-2-amino-4-halobutyric acid derivative enantiomeric mixture to 30 mL/g D-2-amino-4-halobutyric acid derivative or D, L-2-amino-4-halobutyric acid derivative enantiomeric mixture. The temperature of step a) of the method can be from 0° C. to the reflux temperature of the system.

For any of the methods described herein, the L-2-amino-4-halobutyric acid derivative-L-tartaric acid salt can be an L-2-amino-4-halobutyric acid derivative-L-tartrate with a structure of formula I:

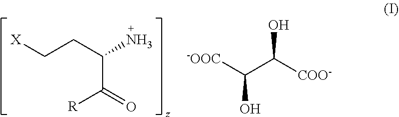

wherein
z is the molar ratio of L-2-amino-4-halobutyric acid derivative to L-tartaric acid, z is from 1 to 2;
X is Cl or Br; and
R is OR¹ or NR²R³, wherein R¹ is a C₁ to C₄ aliphatic hydrocarbon, R² is H or a C₁ to C₄ aliphatic hydrocarbon, and R³ is H or a C₁ to C₄ aliphatic hydrocarbon.

For any of the methods described herein, the D, L-2-amino-4-halobutyric acid derivative enantiomeric mixture can be a mixture of L-2-amino-4-halobutyric acid derivatives and D-2-amino-4-halobutyric acid derivatives with a structure of formula II:

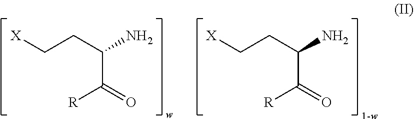

wherein
X is Cl or Br;
R is OR¹ or NR²R³, wherein R¹ is a C₁ to C₄ aliphatic hydrocarbon, R² is H or a C₁ to C₄ aliphatic hydrocarbon, and R³ is H or a C₁ to C₄ aliphatic hydrocarbon; and
w is the molar percentage of L-2-amino-4-halobutyric acid derivatives in the enantiomeric mixture of D, L-2-amino-4-halobutyric acid derivatives.

In the present disclosure, the inventors found that 4-halo-2-aminobutyric acid and 4-halo-2-aminobutyramide are extremely unstable, and the existing resolution technique of D, L-aminobutyric acid or D, L-aminobutyramide using L-tartaric acid as directed by patent applications WO2006103696A2 and CN102060721A cannot be directly used for the resolution of 4-halo-2-aminobutyric acid and 4-halo-2-aminobutyramide. In the course of their research, the inventors were surprised to find that 4-halo-2-aminobutyrate and 4-halo-2-aminobutyramide tartrates were structurally stable and that the tartrates of stable L-2-amino-4-halo-butyric acid derivatives could be obtained after the resolution reaction. L-2-amino-4-halobutyric acid derivative is an important intermediate for the preparation of L-ammonium glufosinate.

The present disclosure also provides a simple and workable method for the preparation of L-2-amino-4-halobutyric acid derivative-L-tartrate.

In the course of studying the resolution of the enantiomeric mixture of D, L-2-amino-4-halobutyric acid derivatives, the inventors were surprised to find that the resolution could be done in one step using the same resolution solvent system according to the methods described herein.

According to method a) described herein, L-2-amino-4-halobutyric acid derivative-L-tartrate can be prepared by a method comprising using D, L-2-amino-4-halobutyric acid derivative enantiomeric mixture as raw material, using L-tartaric acid as the resolution agent, and obtaining L-2-amino-4-halobutyric acid derivative-L-tartrate by resolution reaction, precipitation, and filtration in the same resolution agent system.

According to method b) described herein, L-2-amino-4-halobutyric acid derivative-L-tartrate can be prepared by a method comprising using D, L-2-amino-4-halobutyric acid derivative enantiomeric mixture as raw material, catalyzing by aromatic aldehydes or aromatic aldehydes and organic acids, with L-tartaric acid as the resolution agent, in the same resolution agent system, by dynamic resolution reaction so that the resolution and racemization proceed simultaneously, further comprising precipitation and filtration to obtain L-2-amino-4-halobutyric acid derivative-L-tartrate.

In method a), the main reaction formula for the preparation of L-2-amino-4-halobutyric acid derivative-L-tartrate as described is as follows:

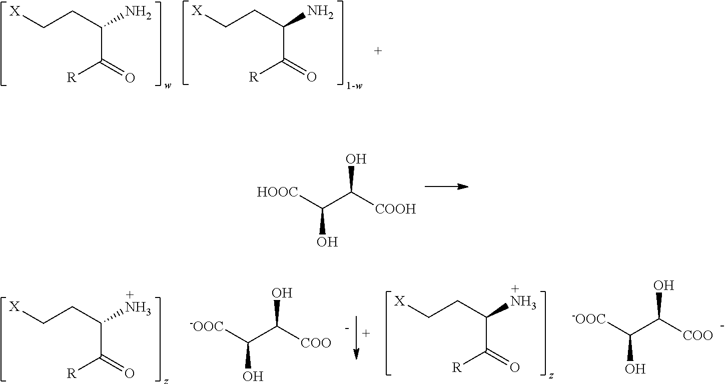

The following are the preferred technical solutions of method a) of the present disclosure.

In method a), the molar ratio of L-2-amino-4-halobutyric acid derivatives to D-2-amino-4-halobutyric acid derivatives is from 0.5 to 1.5:1. When the content of D-2-amino-4-halobutyric acid derivatives in the raw material is too high, the resolution process cannot precipitate solids well, and the products obtained from the resolution system are still mostly of D-configuration, which need to be recrystallized several times to obtain products rich in L-configuration. Therefore, the D, L-2-amino-4-halobutyric acid derivatives of the D-configuration-rich isomers are not suitable for direct disassembly in method a); whereas, when the L-2-amino-4-halobutyric acid derivatives in the feedstock are too high, they can be salified with L-tartaric acid first, and then recrystallized to obtain the L-configuration-rich products. Further preferably, the molar ratio of L-2-amino-4-halobutyric acid derivatives to D-2-amino-4-halobutyric acid derivatives in the raw material is from 1 to 1.5:1.

In method a), the resolving agent used is L-tartaric acid. In a suitable solvent system, the enantiomeric mixture of D, L-2-amino-4-halobutyric acid derivatives reacts with L-tartaric acid to form L-2-amino-4-halobutyric acid derivative-L-tartrate (I) and D-2-amino-4-halobutyric acid derivative-L-tartrate, which are enantiomeric to each other, and L-2-amino-4-halobutyric acid derivative-L-tartrate (I) can be further isolated by using their different solubility in the solvent system.

In method a), the molar ratio of the enantiomeric mixture of D, L-2-amino-4-halobutyric acid derivatives to L-tartaric acid is from 1:0.2 to 1.5. When the amount of L-tartaric acid is low, the product yield is low; when the amount of L-tartaric acid is large, the resolution effect is not significantly changed, and the resolution agent is wasted, which increases the difficulty of post-processing and causes unnecessary pollution. Further preferably, the molar ratio of D, L-2-amino-4-halobutyric acid derivative enantiomeric mixture to L-tartaric acid is from 1:0.2 to 1.

In method a), the resolution solvent is a mixture of C₁ to C₄ lower alcohol and water or C₁-C₄ lower alcohol; the C₁-C₄ lower alcohol is one or more of methanol, ethanol, n-propanol, isopropanol, n-butanol, isobutanol, and tert-butanol, further preferably methanol or ethanol. The volume percentage of the C₁-C₄ lower alcohol in the mixed solvent composed of the C₁-C₄ lower alcohol and water is from 10% to 99.99%. When the volume percentage of C₁-C₄ lower alcohols is low, the solubility of L-2-amino-4-halobutyric acid derivative-L-tartrate and D-2-amino-4-halobutyric acid derivative-L-tartrate are both very large, and their solubility also varies greatly, so the resolution reaction yield is low. As the alcohol volume percentage increases, the solubility of L-2-amino-4-halobutyric acid derivative-L-tartrate (formula I structure) and D-2-amino-4-halobutyric acid derivative-L-tartrate as well as the solubility difference became smaller, and the product optical purity was poor and the yield was low. Further preferably, the volume percentage of C₁-C₄ lower alcohols in the solvent mixture consisting of C₁-C₄ lower alcohols and water is from 70 to 95%.

In method a), the volume of the resolution solvent is from 1 mL/g raw material to 30 mL/g raw material, that is, lg of D, L-2-amino-4-halobutyric acid derivative enantiomeric mixture requires 1.5 mL-30 mL of resolution solvent. When 1 g of the enantiomeric mixture of D, L-2-amino-4-halobutyric acid derivatives uses a resolution solvent less than 1.5 mL, in addition to most of the L-2-amino-4-halobutyric acid derivative-L-tartrate being in the precipitated solid, there is also part of the D-2-amino-4-halobutyric acid derivative-L-tartrate; the obtained products have poor optical purity. When 1 g of the enantiomeric mixture of D, L-2-amino-4-halobutyric acid derivatives uses a resolution solvent more than 30 mL, only a small amount of L-2-amino-4-halobutyric acid derivative-L-tartrate is precipitated, and the product yield is low. Further preferably, the volume of the resolution solvent is from 3 mL to 8 mL/g raw material, i.e., 1 g of the enantiomeric mixture of D, L-2-amino-4-halobutyric acid derivatives requires 3 mL to 8 mL of resolution solvent.

In method a), the temperature of the resolution reaction is 0° C. to the reflux temperature of the system. The solubility of L-2-amino-4-halobutyric acid derivative-L-tartrate (I) and D-2-amino-4-halobutyric acid derivative-L-tartrate and their relative solubility differences vary with temperature. At high temperature, the solubility difference between the two is large, and a product with high optical purity can be obtained, but at high temperature conditions the solubility of the two is large, so the yield is extremely low. At low temperature, the solubility difference between the two is small, and the product obtained has poor optical purity. Further preferably, the temperature of the resolution reaction is from 20 to 65° C.

In method b), the main reaction formula for the preparation of L-2-amino-4-halobutyric acid derivative-L-tartrate as described is as follows:

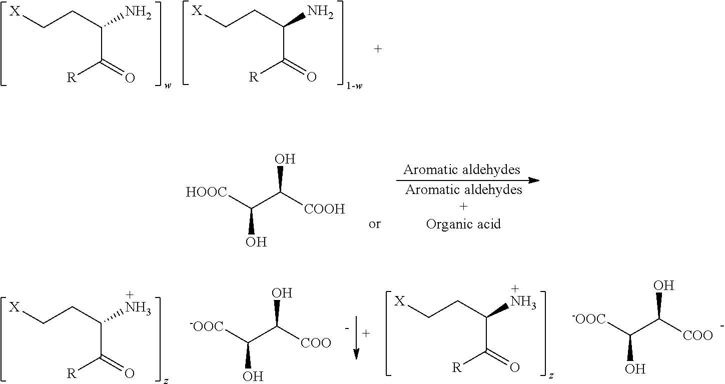

The following are the preferred technical solutions of method b) of the present disclosure.

In method b), the molar ratio of L-2-amino-4-halobutyric acid derivatives to D-2-amino-4-halobutyric acid derivatives is from 0 to 1.5:1. Since the reaction described in b) is a dynamic resolution reaction, when the content of D-2-amino-4-halobutyric acid derivatives in the raw material is too high, using L-tartaric acid as the resolving agent, the resolution and racemization are carried out simultaneously under the catalysis of aromatic aldehydes or the co-catalysis of aromatic aldehydes and organic acids in the same resolution system. This results in the conversion of the D-configuration to the L-configuration in the reaction system and the formation of the corresponding L-2-amino-4-halobutyric acid derivative-L-tartrate, and the difference in solubility between L-configuration tartrate and D-configuration tartrate in the solvent is used to precipitate the product rich in L-configuration. When the content of L-2-amino-4-halobutyric acid derivative in the raw material is too high, it can first form a salt with L-tartrate and then directly refine it by recrystallization to obtain L-2-amino-4-halobutyric acid derivative-L tartaric acid salt. Further preferably, the molar ratio of L-2-amino-4-halobutyric acid derivative to D-2-amino-4-halobutyric acid derivative in the raw material is from 0 to 1:1.

In method b), the molar ratio of the enantiomeric mixture of D, L-2-amino-4-halobutyric acid derivatives to L-tartaric acid is from 1:0.3 to 1.5. In the process of dynamic resolution, with the continuous progress of racemization and resolution reactions, the content of L-configuration tartrate in the system is always kept low. Using the solubility difference between L-configuration tartrate and D-configuration tartrate in the solvent system, the L-configuration tartrate is continuously precipitated, and the dissolved tartaric acid in the system is consumed. When the amount of L-tartaric acid is low, the L-configuration salt formation will be insufficient and the yield will be too low. When the amount of L-tartaric acid is large, the resolution effect is not significantly changed, and the resolution agent is wasted, which increases the difficulty of post-processing and causes unnecessary pollution. Further preferably, the molar ratio of D, L-2-amino-4-halobutyric acid derivative enantiomeric mixture to L-tartaric acid is from 1:0.5 to 1.2.

In method b), the aromatic aldehyde is one or more of benzaldehyde, salicylaldehyde, 5-nitrosalicylaldehyde or 3,5-dinitrosalicylaldehyde. The use of aromatic aldehydes with a hydroxyl group at the 2-position of the aldehyde group in dynamic resolution can better catalyze the racemization reaction. Further preferably, the aromatic aldehyde is salicylic aldehyde.

In method b), the molar ratio of the D, L-2-amino-4-halobutyric acid derivative enantiomeric mixture to the aromatic aldehyde is from 1:0.01 to 0.5. When the amount of aromatic aldehydes is large, it does not significantly improve the catalytic reaction, wastes raw materials, and is cumbersome for subsequent treatment. When the amount of aromatic aldehydes is small, it does not catalyze the reaction well and the product yield is low. Further preferably, the molar ratio of enantiomeric mixture of D, L-2-amino-4-halobutyric acid derivatives to aromatic aldehydes is from 1:0.01 to 0.4.

In method b), the catalysis by aromatic aldehydes or aromatic aldehydes and organic acids, with L-tartaric acid as the resolution agent, in the same resolution agent system, by dynamic resolution reaction, so that the resolution and racemization proceed simultaneously. In this case, the reaction catalyzed by aromatic aldehyde alone can achieve the desired effect, but the reaction time is long; when using aromatic aldehyde co-catalyzed with organic acids, the reaction time can be substantially shortened and the reaction efficiency improved.

In method b), the organic acid is one or more of formic acid, acetic acid, and propionic acid. Further preferred is acetic acid.

In method b), the molar ratio of the enantiomeric mixture of D, L-2-amino-4-halobutyric acid derivatives to organic acid is from 1:0.1 to 5. When the amount of organic acid is large, it does not significantly improve the catalytic reaction, wastes raw materials and has no practical significance; when the amount of organic acid is small, it cannot achieve the expected catalytic efficiency. Further preferably, the molar ratio is from 1:0.3 to 1.5.

In method b), the resolution solvent is a mixture of C₁ to C₄ lower alcohol and water or C₁-C₄ lower alcohol; the C₁-C₄ lower alcohol is one or more of methanol, ethanol, n-propanol, isopropanol, n-butanol, isobutanol, tert-butanol, further preferably methanol or ethanol. The volume percentage of the C₁-C₄ lower alcohol in the mixed solvent composed of the C₁-C₄ lower alcohol and water is from 80% to 99.99%. When the water content in the resolution solvent is high, the racemization reaction cannot occur well, and the solubility of L-2-amino-4-halobutyric acid derivative-L-tartrate and D-2-amino-4-halobutyric acid derivative-L-tartrate is very large, the solubility difference is also very large, and the yield is low. Further preferably, the volume percentage of C₁-C₄ lower alcohols in the solvent mixture consisting of C₁-C₄ lower alcohols and water is from 85 to 99.99%.

In method b), the volume of the resolution solvent is from 1 mL/g raw material to 30 mL/g raw material, that is, lg of D, L-2-amino-4-halobutyric acid derivative enantiomeric mixture requires 1.5 mL-30 mL of resolution solvent. When 1 g of the enantiomeric mixture of D, L-2-amino-4-halobutyric acid derivatives uses a resolution solvent less than 1.5 mL, in addition to most of the L-2-amino-4-halobutyric acid derivative-L-tartrate being in the precipitated solid, there is also part of the D-2-amino-4-halobutyric acid derivative-L-tartrate; the obtained products have poor optical purity. When 1 g of the enantiomeric mixture of D, L-2-amino-4-halobutyric acid derivatives uses a resolution solvent more than 30 mL, only a small amount of L-2-amino-4-halobutyric acid derivative-L-tartrate is precipitated, and the product yield is low. Further preferably, the volume of the resolution solvent is from 3 mL to 8 mL/g raw material, i.e., 1 g of the enantiomeric mixture of D, L-2-amino-4-halobutyric acid derivatives requires 3 mL to 8 mL of resolution solvent.

In method b), the temperature of the resolution reaction is 0° C. to the reflux temperature of the system. The solubility of L-2-amino-4-halobutyric acid derivative-L-tartrate (I) and D-2-amino-4-halobutyric acid derivative-L-tartrate and their relative solubility differences vary with temperature. At high temperature, the solubility difference between the two is large, and a product with high optical purity can be obtained, but at high temperature conditions the solubility of the two is large, so the yield is extremely low. At low temperature, the solubility difference between the two is small, and the product obtained has poor optical purity. Further preferably, the temperature of the resolution reaction is from 20 to 65° C.

The present invention has the following advantages:

The L-2-amino-4-halobutyric acid derivative-L-tartrate of the present disclosure is a type of chiral intermediate with stable properties, convenient transportation and storage, and can be converted into L-2-amino-4-halobutyric acid derivatives through simple steps, further used in the synthesis of L-glufosinate.

The preparation method of L-2-amino-4-halobutyric acid derivative-L-tartrate of the present disclosure is suitable for raw materials with various chiral contents.

The preparation method of L-2-amino-4-halobutyric acid derivative-L-tartrate of the present disclosure, which adopts the completion of the resolution reaction in the same resolution solvent system, is simple in preparation and operation, and is a low cost process for the preparation of chiral aminobutyric acid blocks, which is a key intermediate of L-glufosinate.

Having described the invention in detail, it will be apparent that modifications and variations are possible without departing from the scope of the invention defined in the appended claims.

### EXAMPLES

The following non-limiting examples are provided to further illustrate the present invention.

The present invention is further elucidated hereinafter in connection with specific embodiments, it being understood that these embodiments are intended only to illustrate the invention and not to limit the scope of the invention, and that after reading the invention, various modifications of the invention in equivalent form by those skilled in the art fall within the scope defined by the claims of the invention.

### Example 1

In a 250 mL three-necked flask under stirring, D, L-2-amino-4-chlorobutyric acid ethyl ester enantiomeric mixture (optical purity D:L=50:50) 16.56 g (0.1 mol) was added to ethanol 100 mL and water 20 mL, and then L-tartaric acid 7.5 g (0.05 mol) was added. This was stirred at 70° C. for 10 min to dissolve clear, slowly cooled down within 4 hours to 20° C., and filtered. The filter cake was dried to obtain L-2-amino-4-chlorobutyric acid ethyl ester-L-tartrate crude 9.86 g, yield 40.98%, optical purity D:L=6.4:93.6.

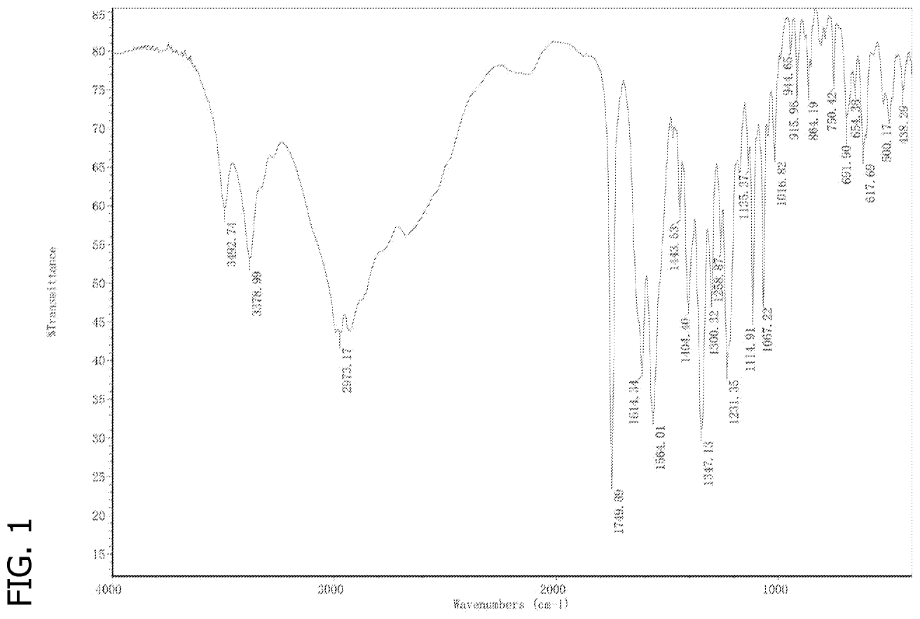

IR (KBr) ν (cm⁻¹) 3493, 3379, 2973, 1750, 1614, 1564, 1404, 1347, 1231, 1115, 1067, 1016, 915, 864, 617, as shown in FIG. 1.

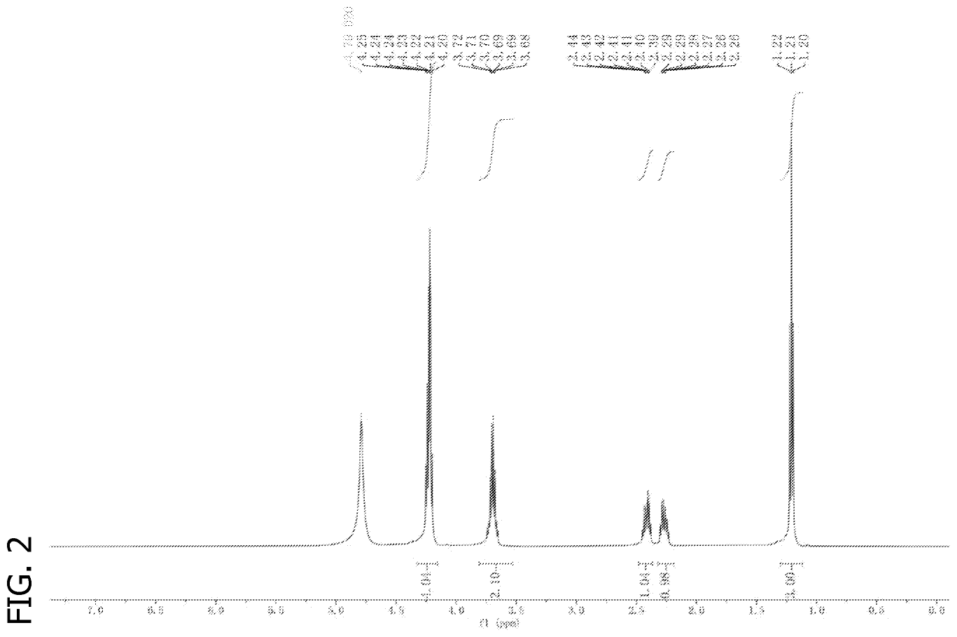

¹H NMR (500 MHz, D₂O) δ 4.33-4.16 (m, J=5.1 Hz, 4H), 3.81-3.53 (m, 2H), 2.48-2.36 (m, 1H), 2.32-2.19 (m, 1H), 1.21 (t, J=7.2 Hz, 3H), as shown in FIG. 2.

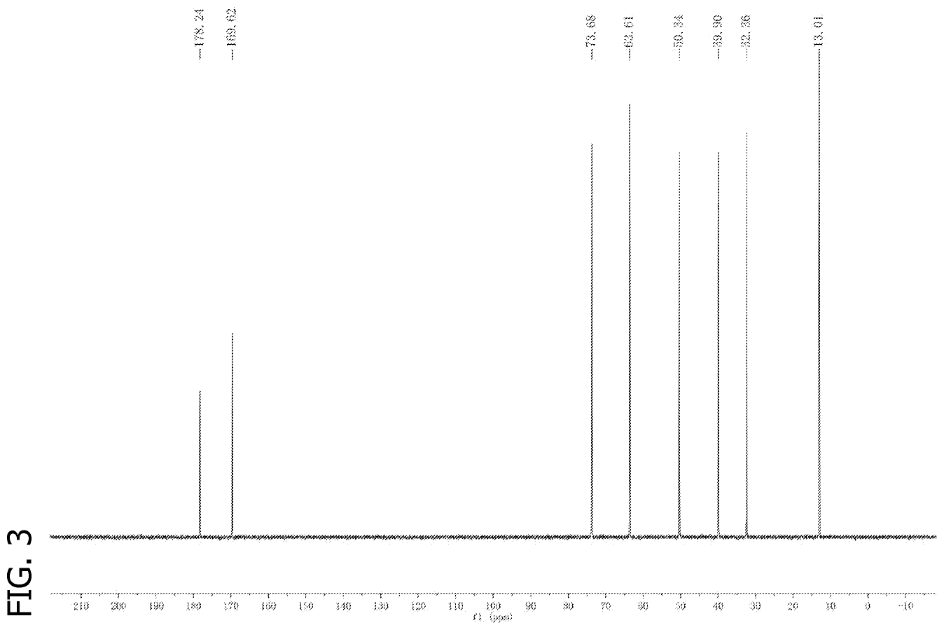

¹³C NMR (126 MHz, D₂O) δ 178.24, 169.62, 73.68, 63.61, 50.34, 39.90, 32.36, 13.01, as shown in FIG. 3.

### Example 2

In a 250 mL three-necked flask under stirring, D, L-2-amino-4-chlorobutyric acid ethyl ester enantiomeric mixture (optical purity D:L=50:50) 24.84 g (0.15 mol) was added to ethanol 150 mL and water 10 mL, and then L-tartaric acid 6.75 g (0.045 mol) was added. This was stirred at 60° C. for 10 min to dissolve clear, slowly cooled down within 4 hours to 20° C., and filtered. The filter cake was dried to obtain L-2-amino-4-chlorobutyric acid ethyl ester-L-tartrate crude 13.12 g, yield 36.35%, optical purity D:L=3.2:96.8.

### Example 3

In a 250 mL three-necked flask under stirring, D, L-2-amino-4-chlorobutyric acid ethyl ester enantiomeric mixture (optical purity D:L=50:50) 20 g (0.12 mol) was added to ethanol 100 mL and salicylaldehyde 4 g, and then L-tartaric acid 9.96 g (0.066 mol) and acetic acid 10 g were added. This was stirred at 60° C. for 3 hours, slowly cooled down within 5 hours to 20° C., and filtered. The filter cake was dried to obtain L-2-amino-4-chlorobutyric acid ethyl ester-L-tartrate crude 20.39 g, yield 70.17%, optical purity D:L=6.2:93.8.

### Example 4

In a 250 mL three-necked flask under stirring, D, L-2-amino-4-chlorobutyric acid ethyl ester enantiomeric mixture (optical purity D:L=80:20) 20 g (0.12 mol) was added to ethanol 100 mL and salicylaldehyde 4 g, and then L-tartaric acid 9.96 g (0.066 mol) and acetic acid 10 g were added. This was stirred at 60° C. for 3 hours, slowly cooled down within 5 hours to 20° C., and filtered. The filter cake is dried to obtain L-2-amino-4-chlorobutyric acid ethyl ester-L-tartrate crude 18.25 g, yield 62.8%, optical purity D:L=4.8:95.2.

### Example 5

In a 250 mL three-necked flask under stirring, D, L-2-amino-4-chlorobutyric acid ethyl ester enantiomeric mixture (optical purity D:L=50:50) 20 g (0.12 mol), was added to ethanol 100 mL and salicylaldehyde 4 g, and then L-tartaric acid 9.96 g (0.066 mol) and acetic acid 10 g were added. This was stirred at 60° C. for 10 hours, slowly cooled down within 5 hours to 20° C., and filtered. The filter cake was dried to obtain L-2-amino-4-chlorobutyric acid ethyl ester-L-tartrate crude 14.38 g, yield 49.48%, optical purity D:L=5.3:94.7.

### Example 6

In a 250 mL three-necked flask under stirring, D, L-2-amino-4-bromobutyric acid ethyl ester enantiomeric mixture (optical purity D:L=50:50) 21 g (0.1 mol) was added to ethanol 100 mL and water 10 mL, and then L-tartaric acid 7.5 g (0.05 mol) was added. This was stirred at 60° C. for 10 min to dissolve clear, slowly cooled down within 4 hours to 20° C., and filtered. The filter cake was dried to obtain L-2-amino-4-bromobutyric acid ethyl ester-L-tartrate crude 10.55 g, yield 37%, optical purity D:L=6.1:93.9.

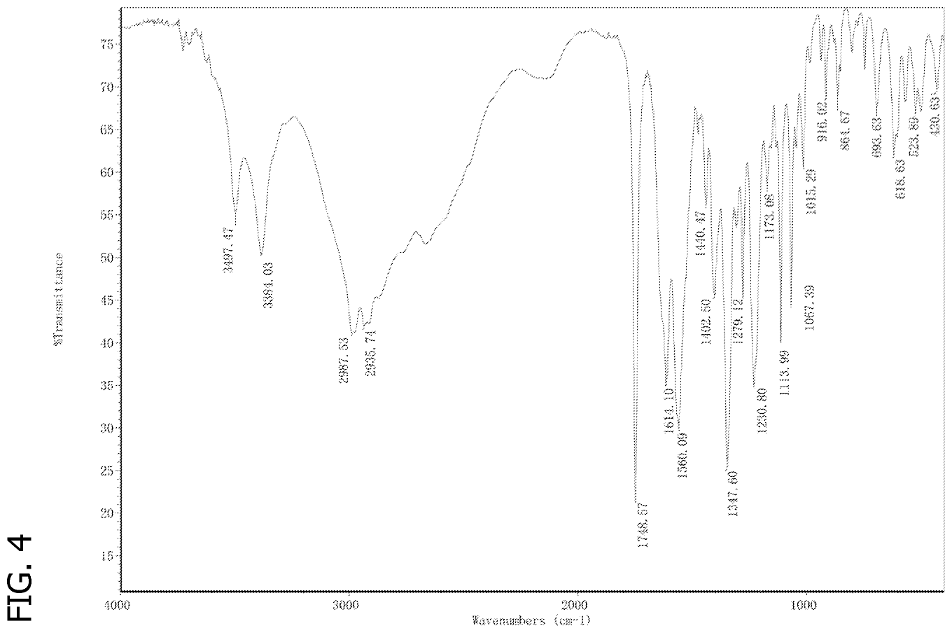

IR (KBr) ν (cm⁻¹) 3497, 3384, 2988, 2936, 1749, 1614, 1560, 1348, 1279, 1231, 1114, 1067, 865, 694, 619, 524, as shown in FIG. 4.

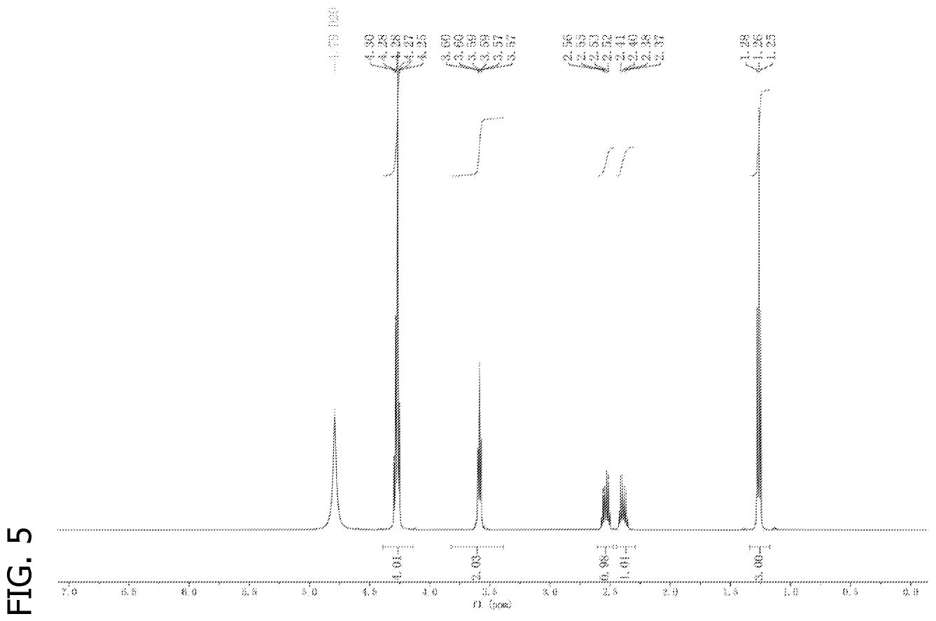

¹H NMR (500 MHz, D₂O) δ 4.39-4.14 (m, 4H), 3.83-3.39 (m, 2H), 2.61-2.47 (m, 1H), 2.45-2.29 (m, 1H), 1.26 (t, J=7.2 Hz, 3H), as shown in FIG. 5.

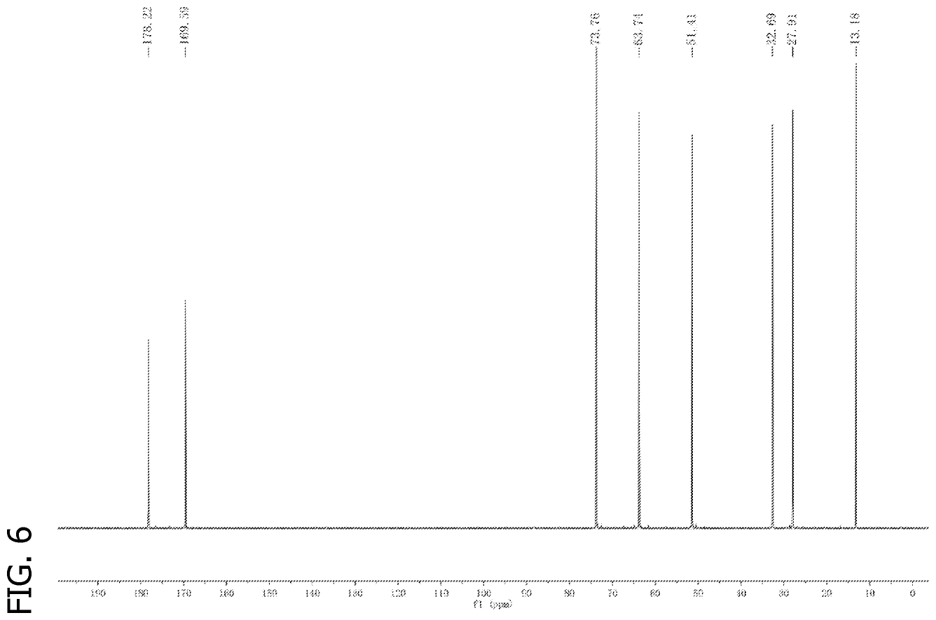

¹³C NMR (126 MHz, D₂O) δ 178.22, 169.59, 73.76, 63.74, 51.41, 32.69, 27.91, 13.18, as shown in FIG. 6.

### Example 7

In a 250 mL three-necked flask under stirring, D, L-2-amino-4-bromobutyric acid ethyl ester enantiomeric mixture (optical purity D:L=50:50) 3.15 g (0.15 mol) was added to ethanol 150 mL and salicylaldehyde 5 g, and then L-tartaric acid 11.25 g (0.075 mol) and acetic acid 15 g were added. This was stirred at 60° C. for 18 hours, slowly cooled down within 3 hours to 20° C., and filtered. The filter cake was dried to obtain L-2-amino-4-bromobutyric acid ethyl ester-L-tartrate crude 27.8 g, yield 65%, optical purity D:L=2.7:97.3.

### Example 8

In a 250 mL three-necked flask under stirring, D, L-2-amino-4-chlorobutyric acid methyl ester enantiomeric mixture (optical purity D:L=50:50) 15.2 g (0.1 mol) was added to methanol 100 mL, and then L-tartaric acid 7.5 g (0.05 mol) was added. This was stirred at 60° C. for 10 min to dissolve clear, slowly cooled down within 5 hours to 20° C., and filtered. The filter cake was dried to obtain L-2-amino-4-chlorobutyric acid methyl ester-L-tartrate crude 6.93 g, yield 30.5%, optical purity D:L=4.3:95.7.

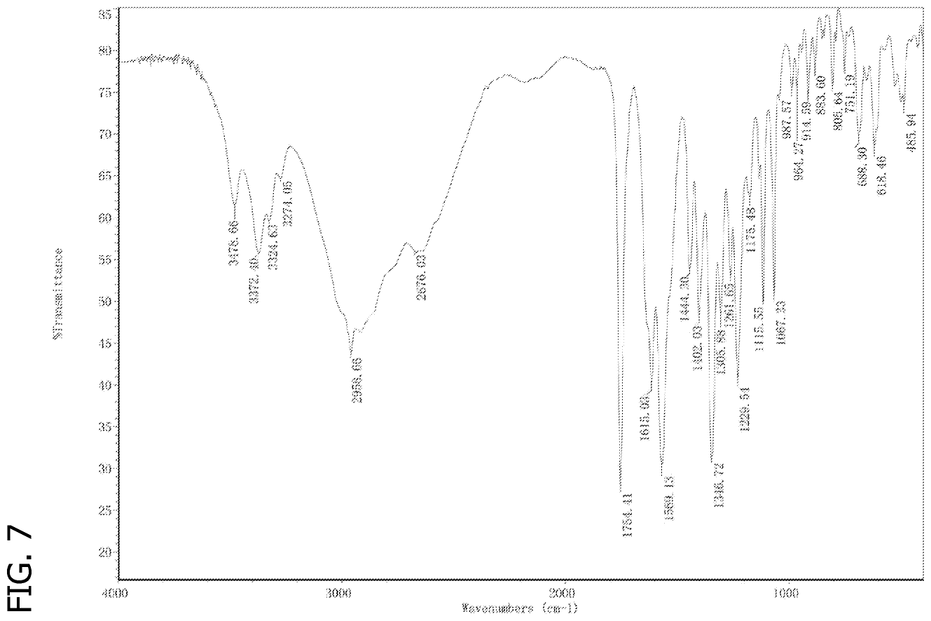

IR (KBr) ν (cm⁻¹) 3479, 3372, 3325, 2959, 2676, 1754, 1615, 1569, 1347, 1306, 1230, 1116, 1067, 806, 688, 618, as shown in FIG. 7.

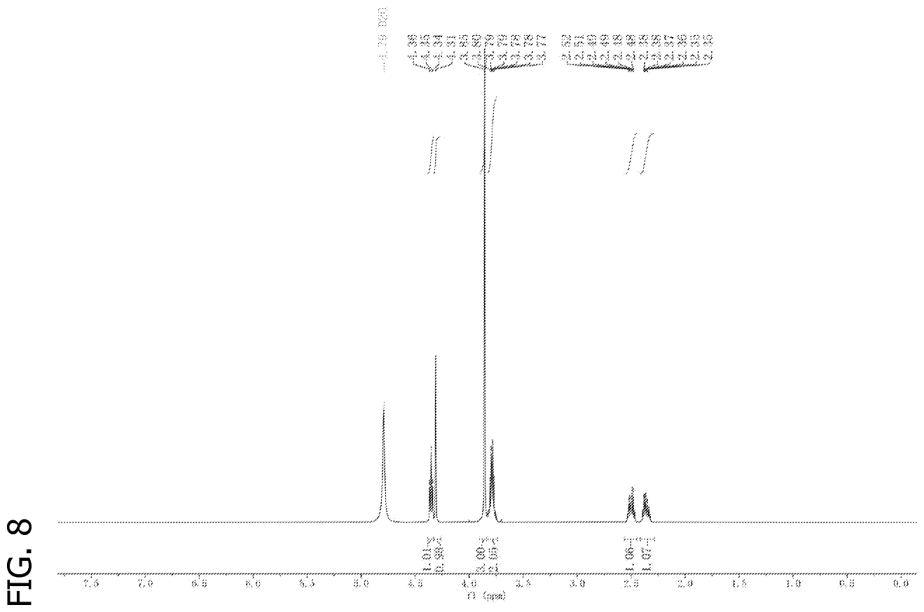

¹H NMR (500 MHz, D₂O) δ 4.35 (t, J=6.6 Hz, 1H), 4.31 (s, 1H), 3.85 (s, 3H), 3.82-3.73 (m, 2H), 2.56-2.45 (m, 1H), 2.42-2.28 (m, 1H), as shown in FIG. 8.

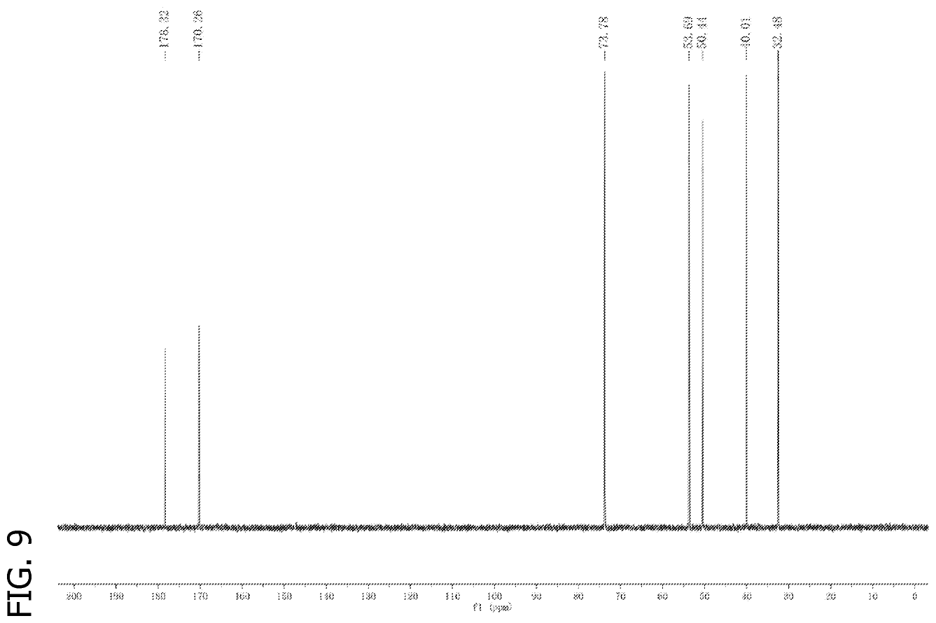

¹³C NMR (126 MHz, D₂O) δ 178.32, 170.26, 73.78, 53.69, 50.44, 40.01, 32.48, as shown in FIG. 9.

### Example 9

In a 250 mL three-necked flask under stirring, D, L-2-amino-4-chlorobutyric acid methyl ester enantiomeric mixture (optical purity D:L=50:50) 20 g (0.132 mol) was added to methanol 80 mL and salicylaldehyde 4 g, and then L-tartaric acid 9.9 g (0.066 mol) and acetic acid 10 g were added. This was stirred at 50° C. for 3 hours, slowly cooled down within 3 hours to 20° C., and filtered. The filter cake was dried to obtain L-2-amino-4-chlorobutyric acid methyl ester-L-tartrate crude 16.3 g, yield 54.5%, optical purity D:L=4.8:95.2.

### Example 10

In a 250 mL three-necked flask under stirring, D, L-2-amino-4-chlorobutyric acid isopropyl ester enantiomeric mixture (optical purity D:L=50:50) 17.96 g (0.1 mol) was added to isopropyl alcohol 80 mL and water 16 g, and then L-tartaric acid 15 g (0.1 mol) was added. This was stirred at 50° C. for 10 min to dissolve clear, slowly cooled down within 3 hours to 30° C., and filtered. The filter cake was dried to obtain L-2-amino-4-chlorobutyric acid isopropyl ester-L-tartrate crude 12.53 g, yield 38%, optical purity D:L=1.3:98.7.

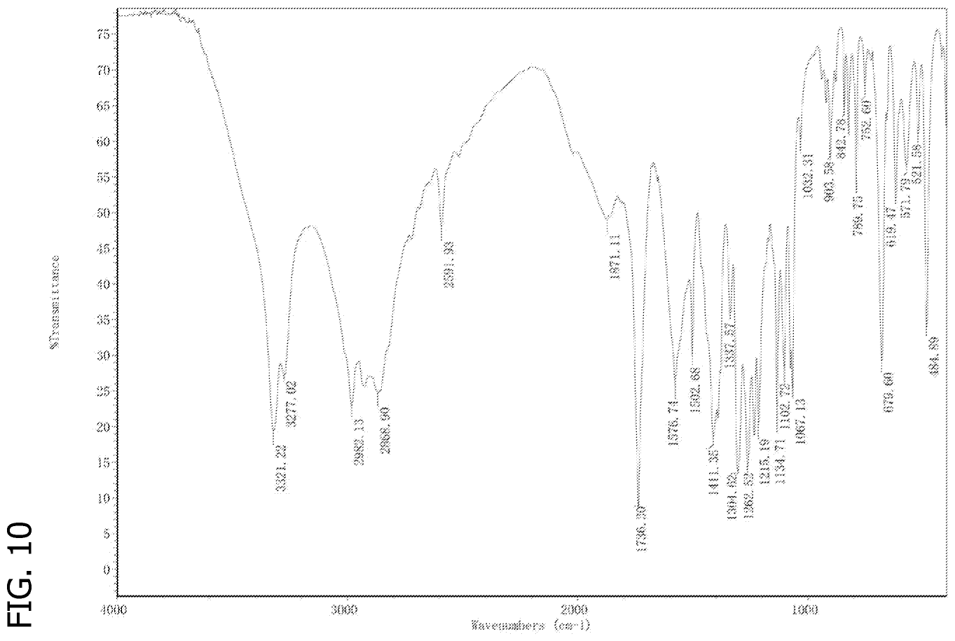

IR (KBr) ν (cm⁻¹) 3321, 3277, 2982, 2869, 2592, 1871, 1736, 1577, 1503, 1411, 1305, 1263, 1215, 1103, 1067, 904, 790, 680, 485, as shown in FIG. 10.

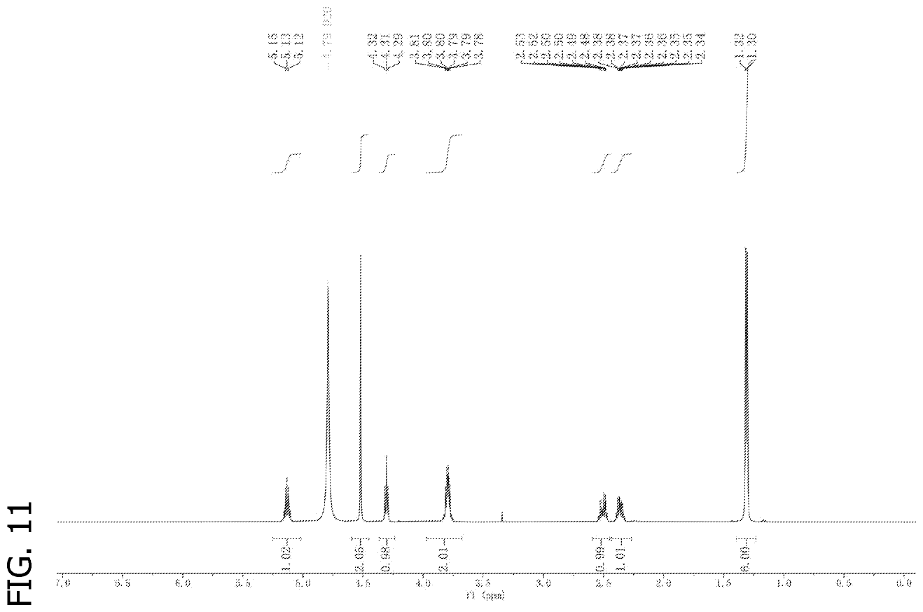

¹H NMR (500 MHz, D₂O) δ 5.25-5.02 (m, 1H), 4.52 (s, 2H), 4.31 (t, J=6.6 Hz, 1H), 3.97-3.68 (m, 2H), 2.59-2.45 (m, 1H), 2.44-2.27 (m, 1H), 1.31 (d, 6H), as shown in FIG. 11.

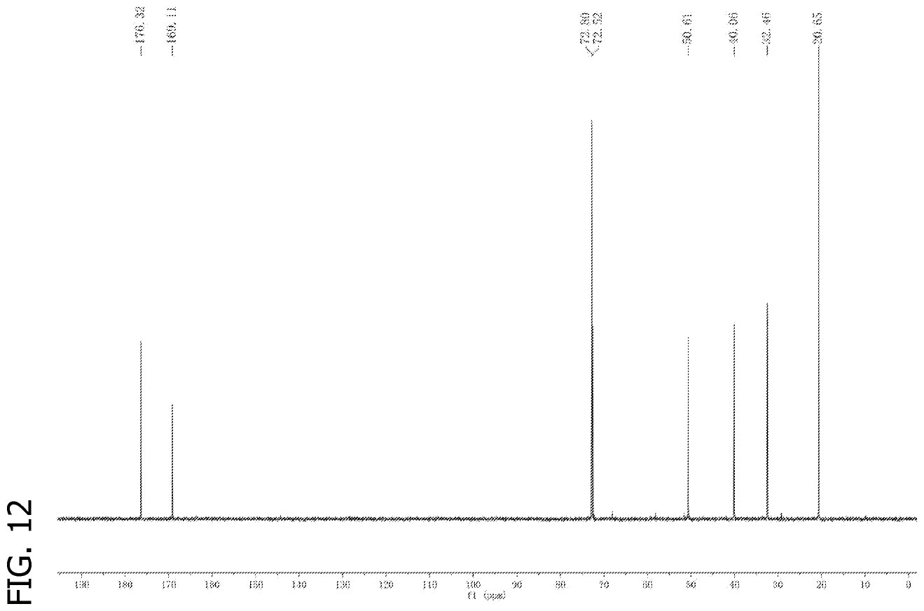

¹³C NMR (126 MHz, D₂O) δ 176.32, 169.11, 72.80, 72.52, 50.61, 40.06, 32.46, 20.65, as shown in FIG. 12.

### Example 11

In a 250 mL three-necked flask under stirring, D, L-2-amino-4-chlorobutyric acid isopropyl ester enantiomeric mixture (optical purity D:L=50:50) 25.15 g (0.14 mol) was added to isopropyl alcohol 120 mL and salicylaldehyde 5 g, and then L-tartaric acid 21 g (0.14 mol) and acetic acid 10 g were added. This was stirred at 55° C. for 3 hours, slowly cooled down within 3 hours to 30° C., and filtered. The filter cake was dried to obtain L-2-amino-4-chlorobutyric acid isopropyl ester-L-tartrate crude 30.2 g, yield 65.4%, optical purity D:L=2.9:97.1.

### Example 12

In a 250 mL three-necked flask under stirring at 5° C. or less, in sequence D, L-2-amino-4-chlorobutyramide enantiomeric mixture (optical purity D:L=50:50) 13.66 g (0.1 mol), methanol 50 mL, and water 10 g were combined, and then L-tartaric acid 7.5 g (0.05 mol) was added. After stirring below 5° C. for 2 hours, the temperature was slowly increased to 60° C. and stirred to dissolve clear, slowly cooled down within 4 hours to 20° C., and filtered. The filter cake was dried to obtain L-2-amino-4-chlorobutyramide-L-tartrate crude 8.8 g, yield 41.6%, optical purity D:L=5.6:94.4.

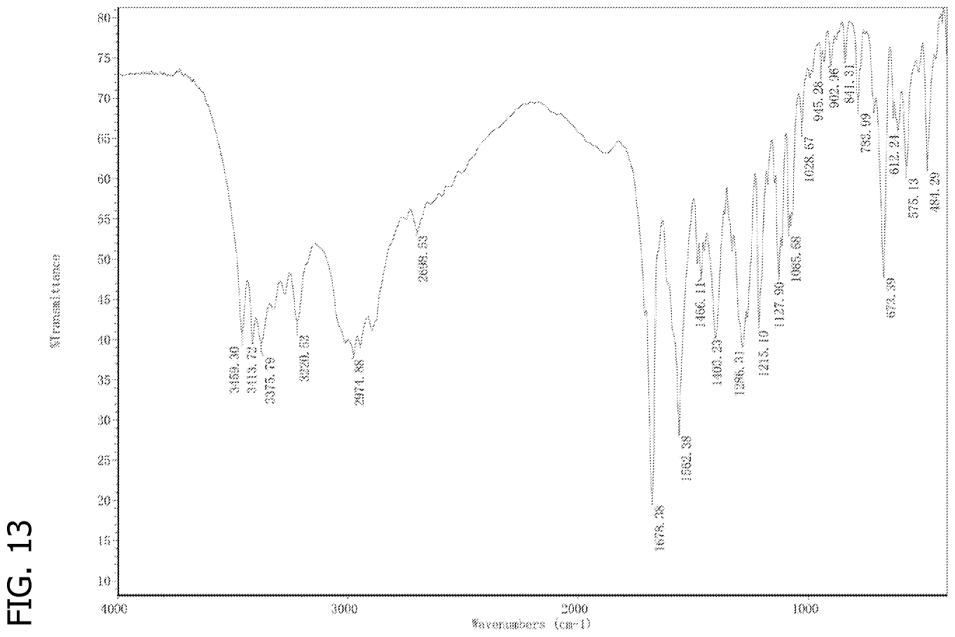

IR (KBr) ν (cm⁻¹) 3459, 3414, 3376, 3221, 2975, 2699, 1678, 1562, 1403, 1286, 1215, 1128, 1086, 784, 673, 575, as shown in FIG. 13.

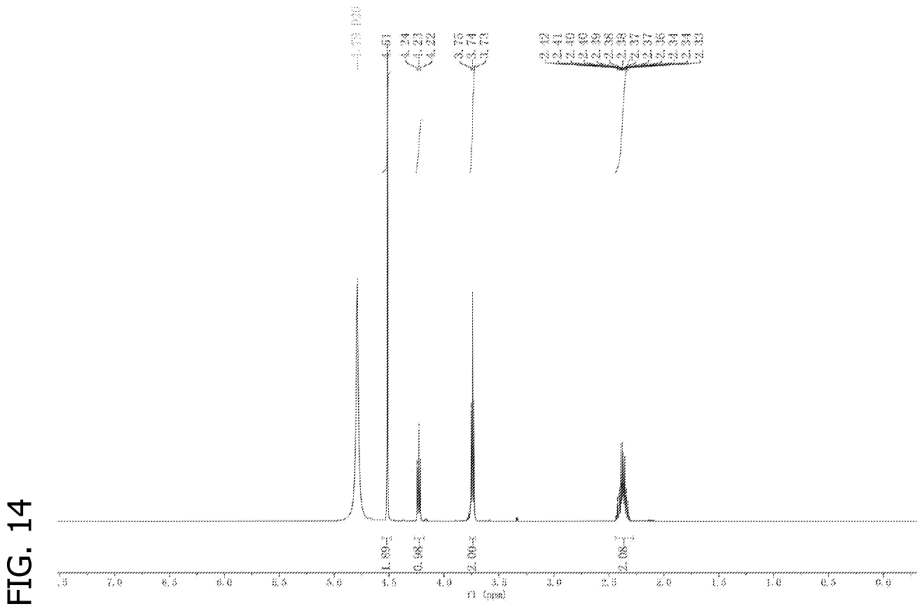

¹H NMR (500 MHz, D₂O) δ 4.51 (s, 2H), 4.23 (t, J=6.8 Hz, 1H), 3.74 (t, J=6.3 Hz, 2H), 2.45-2.28 (m, 2H), as shown in FIG. 14.

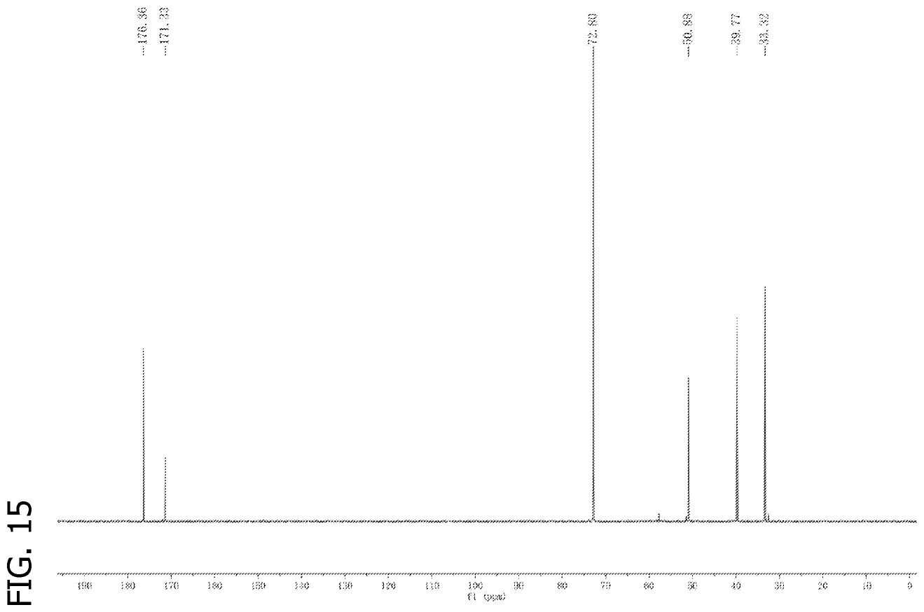

¹³C NMR (126 MHz, D₂O) δ 176.36, 171.33, 72.80, 50.88, 39.77, 33.32, as shown in FIG. 15.

When introducing elements of the present invention or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above products and methods without departing from the scope of the invention, it is intended that all matter contained in the above description and shown in the accompanying drawings shall be interpreted as illustrative and not in a limiting sense.

What is claimed is:

1. An L-2-amino-4-halobutyric acid derivative-L-tartrate with a structure of formula I: 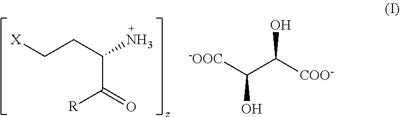
wherein
z is the molar ratio of L-2-amino-4-halobutyric acid derivative to L-tartaric acid, z is from 1 to 2;
X is Cl or Br; and
R is OR¹ or NR²R³, wherein R¹ is a C₁ to C₄ aliphatic hydrocarbon, R² is H or a C₁ to C₄ aliphatic hydrocarbon, and R³ is H or a C₁ to C₄ aliphatic hydrocarbon.

2. A method of preparing a L-2-amino-4-halobutyric acid derivative-L-tartaric acid salt, the method comprising:
a) mixing a D, L-2-amino-4-halobutyric acid derivative enantiomeric mixture, L-tartaric acid, and a solvent to facilitate a resolution reaction and produce a resolution reaction mixture comprising L-2-amino-4-halobutyric acid derivative-L-tartrate;
b) precipitating the L-2-amino-4-halobutyric acid derivative-L-tartrate; and
c) separating the L-2-amino-4-halobutyric acid derivative-L-tartrate from the resolution reaction mixture.

3. The method of claim 2, wherein the molar ratio of L-2-amino-4-halobutyric acid derivatives to D-2-amino-4-halobutyric acid derivatives in the D, L-2-amino-4-halobutyric acid derivative enantiomeric mixture is from 0.5 to 1.5:1.

4. The method of claim 2, wherein the molar ratio of the enantiomeric mixture of D, L-2-amino-4-halobutyric acid derivatives to L-tartaric acid is from 1:0.2 to 1.5.

5. The method of claim 2, wherein the solvent is selected from one or more C₁ to C₄ lower alcohols or a mixture of one or more C₁ to C₄ lower alcohols with water;
the C₁ to C₄ lower alcohol being one or more of methanol, ethanol, n-propanol, isopropanol, n-butanol, isobutanol, and tert-butanol; and
the mixture of one or more C₁ to C₄ lower alcohols with water having a volume percentage of C₁ to C₄ lower alcohols from 10% to 99.99%.

6. The method of claim 2, wherein the volume of the solvent is from 1 mL/g D, L-2-amino-4-halobutyric acid derivative enantiomeric mixture to 30 mL/g D, L-2-amino-4-halobutyric acid derivative enantiomeric mixture.

7. A method of preparing a L-2-amino-4-halobutyric acid derivative-L-tartaric acid salt, the method comprising:
a) mixing a D-2-amino-4-halobutyric acid derivative or a D, L-2-amino-4-halobutyric acid derivative enantiomeric mixture, L-tartaric acid, aromatic aldehydes or aromatic aldehydes and organic acids, and a solvent to facilitate a dynamic resolution reaction and produce a resolution reaction mixture comprising L-2-amino-4-halobutyric acid derivative-L-tartrate;
b) precipitating the L-2-amino-4-halobutyric acid derivative-L-tartrate; and
c) separating the L-2-amino-4-halobutyric acid derivative-L-tartrate from the resolution reaction mixture.

8. The method of claim 7, wherein the molar ratio of L-2-amino-4-halobutyric acid derivatives to D-2-amino-4-halobutyric acid derivatives in the D, L-2-amino-4-halobutyric acid derivative enantiomeric mixture is from 0 to 1.5:1.

9. The method of claim 7, wherein the molar ratio of the D-2-amino-4-halobutyric acid derivative or the enantiomeric mixture of D, L-2-amino-4-halobutyric acid derivatives to L-tartaric acid is from 1:0.3 to 1.5.

10. The method of claim 7, wherein the aromatic aldehydes are one or more of benzaldehyde, salicylaldehyde, 5-nitrosalicylaldehyde, and 3,5-dinitrosalicylaldehyde.

11. The method of claim 7, wherein the molar ratio of the D-2-amino-4-halobutyric acid derivative or the enantiomeric mixture of D, L-2-amino-4-halobutyric acid derivatives to the aromatic aldehyde is from 1:0.01 to 0.5.

12. The method of claim 7, wherein the organic acids are one or more of formic acid, acetic acid, and propionic acid.

13. The method of claim 7, wherein the molar ratio of the D-2-amino-4-halobutyric acid derivative or the enantiomeric mixture of D, L-2-amino-4-halobutyric acid derivatives to the organic acid is from 1:0.1 to 5.

14. The method of claim 7, wherein the solvent is selected from one or more C₁ to C₄ lower alcohols or a mixture of one or more C₁ to C₄ lower alcohols and water;
the C₁ to C₄ lower alcohol being one or more of methanol, ethanol, n-propanol, isopropanol, n-butanol, isobutanol, and tert-butanol; and
the mixture of one or more C₁ to C₄ lower alcohols and water having a volume percentage of C₁ to C₄ lower alcohols from 80% to 99.99%.

15. The method of claim 7, wherein the volume of the solvent is from 1 mL/g D-2-amino-4-halobutyric acid derivative or D, L-2-amino-4-halobutyric acid derivative enantiomeric mixture to 30 mL/g D-2-amino-4-halobutyric acid derivative or D, L-2-amino-4-halobutyric acid derivative enantiomeric mixture.

16. The method of claim 2, wherein the temperature of step a) is from 0° C. to the reflux temperature of the system.

17. The method of claim 2, wherein the L-2-amino-4-halobutyric acid derivative-L-tartaric acid salt is an L-2-amino-4-halobutyric acid derivative-L-tartrate with a structure of formula I: 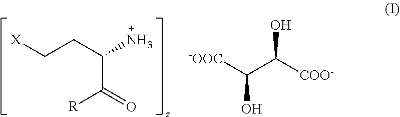
wherein
z is the molar ratio of L-2-amino-4-halobutyric acid derivative to L-tartaric acid, z is from 1 to 2;
X is Cl or Br; and
R is OR¹ or NR²R³, wherein R¹ is a C₁ to C₄ aliphatic hydrocarbon, R² is H or a C₁ to C₄ aliphatic hydrocarbon, and R³ is H or a C₁ to C₄ aliphatic hydrocarbon.

18. The method of claim 2, wherein the D, L-2-amino-4-halobutyric acid derivative enantiomeric mixture is a mixture of L-2-amino-4-halobutyric acid derivatives and D-2-amino-4-halobutyric acid derivatives with a structure of formula II: 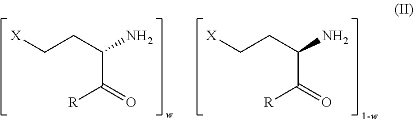
wherein
X is Cl or Br;
R is OR¹ or NR²R³, wherein R¹ is a C₁ to C₄ aliphatic hydrocarbon, R² is H or a C₁ to C₄ aliphatic hydrocarbon, and R³ is H or a C₁ to C₄ aliphatic hydrocarbon; and
w is the molar percentage of L-2-amino-4-halobutyric acid derivatives in the enantiomeric mixture of D, L-2-amino-4-halobutyric acid derivatives.
